(19) United States
(12) Patent Application Publication
(10) Pub. No.: US 2008/0096880 A1
(43) Pub. Date: Apr. 24, 2008

(54) Novel use of imidazotriazinones

(75) Inventors: Helmut Haning, Wuppertal (DE); Erwin Bischoff, Wuppertal (DE); Ulrich Niewohner, Wermelskirchen (DE); Maria Niewohner, Wermelskirchen (DE)

Correspondence Address: Bayer Health Care LLC, 400 Morgan Lane, West Haven, CT, 06516, US

(73) Assignee: Bayer Heakthcare AG, Leverkusen (DE)

(21) Appl. No.: 10/519,129

(22) PCT Filed: Jun. 24, 2003
(86) PCT No.: PCT/EP03/06611

§ 371 Date: Sep. 28, 2005

(30) Foreign Application Priority Data
Jul. 3, 2002 (DE) 102297789

(51) Int. Cl. A61K 31/5377; A61K 31/53; A61P 25/00; A61P 3/00; A61P 9/00; C07D 413/14; C07D 487/04

(52) U.S. Cl. 514/233.2; 514/243; 544/112; 544/184

(57) ABSTRACT

The invention relates to novel uses of imidazo[1,2,4]triazinones for producing a medicament for the treatment and/or prophylaxis of coronary cardiopathy, heart failure, pulmonary hypertension, bladder complaints, prostatic hyperplasia, nitrate-induced tolerance, or diseases of the eye, such as glaucoma, for the treatment or prophylaxis of central, retinal or posterior ciliary artery occlusion, central retinal vein occlusion, optical neuropathy, such as anterior ischaemic optical neuropathy and glaucomatous optical neuropathy, in addition to macular degeneration, diabetes, in particular diabetic gastroparesis, for the treatment of dysperistalsis of the stomach and oesophagus, female infertility, premature contractions, pre-eclampsia, alopecia, psoriasis associated with renal syndrome, cystic fibrosis, cancer and for improving cognition, powers of concentration, learning skills or hypermnesia, in particular if the disorder is a symptom of dementia.

The present invention relates to the use of known imidazotriazinones for producing a medicament for the treatment and/or prophylaxis of coronary heart disease, heart failure, pulmonary hypertension, bladder disorders, prostate hyperplasia, nitrate-induced tolerance, ocular disorders such as glaucoma, for the treatment or prophylaxis of central retinal or posterior cilliary arterial occlusion, central retinal venous occlusion, optic neuropathy such as anterior ischemic optic neuropathy and glaucomatous optic neuropathy, and of macular degeneration, diabetes, especially of diabetic gastroparesis, for the treatment of disorders of the peristalsis of stomach and esophagus, female infertility, premature labor, preeclampsia, alopecia, psoriasis, the renal syndrome, cystic fibrosis, cancer, for improving perception, for improving concentration, for improving learning and/or memory, especially if the impairment is a consequence of dementia.

Imidazotriazinones are described in WO-A 01/64677, the compounds disclosed therein being suitable for the treatment of erectile dysfunction.

German published specification 2811780 describes imidazotriazines as bronchodilators with spasmolytic activity and inhibitory activity on phosphodiesterases which metabolize cyclic adenosine monophosphate (cAMP PDEs, also referred to as PDE III and PDE IV according to the Beavo nomenclature). An inhibitory effect on phosphodiesterases which metabolise cyclic guanosine monophosphate [cGMP PDEs, also referred to as PDE I, PDE II and PDE V according to the nomenclature of Beavo and Reifsnyder (Trends in Pharmacol. Sci. 11, 150-155, 1990)] is not described. In addition, imidazotriazinones which have no substituted aryl radical in the 2 position are described in FR 22 13 058, CH 59 46 71, DE 22 55 172, DE 23 64 076 and EP 000 9384, and are likewise described as bronchodilators with cAMP PDE-inhibitory effect.

WO-A 99/24433 likewise describes imidazotriazinones as cGMP-metabolizing phosphodiesterase inhibitors, but it is obligatory for them to have a sulfonamide group in the position para to the alkoxy group in the phenyl ring.

An increase in the cGMP concentration may lead to therapeutic, antiaggregatory, antithrombotic, antiproliferative, antivasospastic, vasodilating, natriuretic and diuretic effects. It may influence the short- or long-term modulation of vascular and cardiac inotropy, the heart rhythm and cardiac conduction (J.C. Stoclet, T. Keravis, N. Komas and C. Kugnier, Exp. Opin. Invest. Drugs (1995), 4 (11), 1081-1100).

The relaxant effect on smooth muscle leads to a therapeutic improvement in the microcirculation in tissues which comprise cGMP-metabolizing phosphodiesterases.

It has now been found that compounds of the general formula (I)

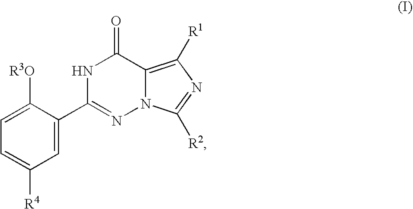

in which

R¹ is (C₁-C₆)-alkyl,

R² is (C₃-C₈)-cycloalkyl or (C₁-C₁₂)-alkyl,

R³ is (C₁-C₆)-alkyl,

R⁴ is a radical of the formulae

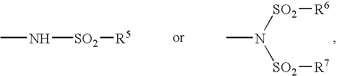

in which

R⁵, R⁶ and R⁷ are identical or different and are vinyl or (C₁-C₆)-alkyl which is optionally substituted up to 3 times, identically or differently, by trifluoromethyl, halogen, (C₁-C₆)-alkoxy or by radicals of the formulae

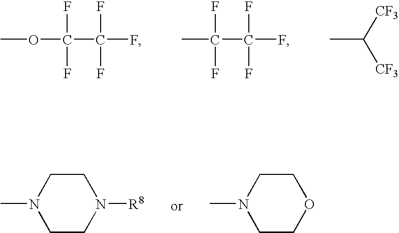

in which

R⁸ is hydrogen or (C₁-C₄)-alkyl,

or

R⁵, R⁶ and/or R⁷ are (C₆-C₁₂)-aryl which is optionally substituted up to 3 times, identically or differently, by halogen, trifluoromethyl, nitro, cyano, carboxyl, (C₁-C₆)-alkyl or (C₁-C₆)-alkoxy,

or

R⁵ is quinolyl or a 5- to 6-membered, aromatic or saturated heterocycle having up to 3 heteroatoms from the series S, N and/or O, which may optionally be substituted, in the case of an N function also via the latter, up to 3 times, identically or differently, by halogen or (C₁-C₆)-alkyl,

or

R⁵ is a radical of the formulae

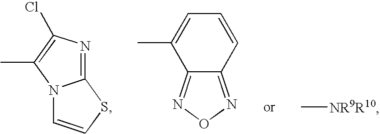

in which

R⁹ and R¹⁰ are identical or different and are hydrogen, (C₁-C₆)-alkyl or phenyl,

or

R⁴ is carboxyl or is a radical of the formulae

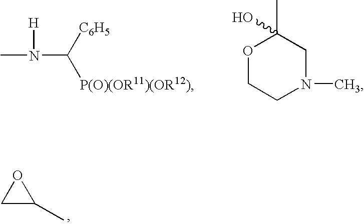

—C—R¹³or —O—R¹⁴,

in which

R¹¹ and R¹² are identical or different and are hydrogen or (C₁-C₄)-alkyl,

R¹³ is (C₁-C₆)-alkyl,

R¹⁴ is (C₁-C₆)-alkyl which is optionally substituted up to 3 times, identically or differently, by hydroxyl, phenyl or by a radical of the formula -NR¹⁵R¹⁶,

in which

R¹⁵ and R¹⁶ are identical or different and are hydrogen, phenyl or (C₁-C₄)-alkyl which in turn may be substituted by phenyl,

or

R⁴ is a radical of the formula —NH—CO—NR¹⁷R¹⁸,

in which

R¹⁷ and R¹⁸ are identical or different and are hydrogen or (C₁-C₆)-alkyl which is optionally substituted by hydroxyl or by a radical of the formulae

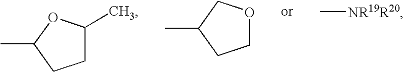

in which

R¹⁹ and R²⁰ are identical or different and are hydrogen, phenyl or (C₁-C₆)-alkyl,

or

R¹⁷ and R¹⁸ form together with the nitrogen atom to which they are bonded a heterocyclic ring of the formulae

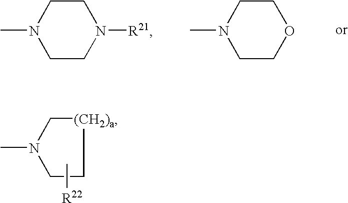

in which

R²¹ is hydrogen or (C₁-C₆)-alkyl,

a is either 1 or 2,

R²² is hydroxyl or (C₁-C₆)-alkyl which is optionally substituted by hydroxyl,

or

R¹⁷ and/or R¹⁸ are (C₆-CI₂)-aryl which is optionally substituted by halogen, trifluoroethyl or by —SCF₃,

or

R¹⁷ is hydrogen and

R¹⁸ is a radical of the formula —SO₂—R²³,

in which

R²³ is (C₁-C₆)-alkyl or (C₆-C₁₂)-aryl which is optionally substituted by halogen,

or is a radical of the formulae

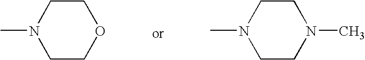

or

R⁴ is a radical of the formula

—NH—CO—R²⁴,

in which

R²⁴ is a radical of the formula

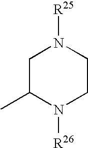

in which

R²⁵ and R²⁶ are identical or different and are hydrogen, (C₁-C₆)-alkyl or (C₁-C₆)-alkoxycarbonyl,

or

R²⁴ is (C₁-C₆)-alkyl which is optionally substituted by (C₆-C₁₂)-aryl which in turn may be substituted by hydroxyl or (C₁-C₆)-alkoxy or

(C₁-C₆)-alkyl is optionally substituted by a radical of the formula —(SO₂)_{b}—R²⁷,

in which

b is either 0 or 1, and

R²⁷ is a radical of the formulae

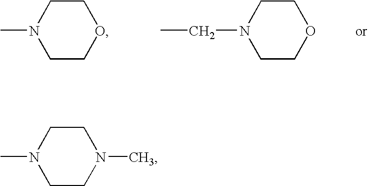

or

R⁴ is (C₁-C₁₂)-alkyl which is optionally substituted up to 3 times, identically or differently, by hydroxyl, azide, phenyl or by radicals of the formulae —NR₂R²⁹, —O—CO—R³⁰ or —P(O){O—[(C₁-C₆)-alkyl]}₂,

in which

R²⁸ and R²⁹ are identical or different, are hydrogen, phenyl or (C₁-C₆)-alkyl which is optionally substituted by hydroxyl, (C₁-C₆)-alkoxy or phenyl,

or

R²⁸ and R²⁹ form together with the nitrogen atom to which they are bonded a heterocyclic ring of the formulae

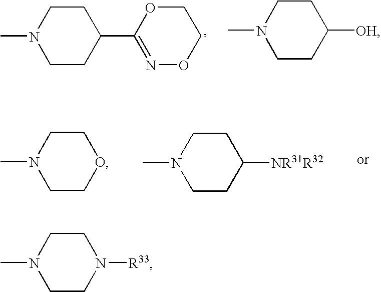

in which

R³¹ and R³² are identical or different and are hydrogen or (C₁-C₆)-alkyl,

R³³ is (C₁-C₆)-alkyl, benzyl, (C₁-C₆)-alkoxycarbonyl, (C₁-C₆)-alkylcarbonyl, carboxyl, pyridyl, pyrimidyl or phenyl which is optionally substituted by (C₁-C₆)-alkoxy,

and

R³⁰ is (C₁-C₆)-alkyl,

or

(C₁-C₁₂)-alkyl is optionally substituted by triazolyl which may in turn be substituted up to twice, identically or differently, by halogen, phenyl, tetrahydrofuranyl, tetrahydropyranyl, (C₁-C₆)-alkoxycarbonyl, aminocarbonyl or by (C₁-C₆)-alkyl, where the latter can optionally be substituted by hydroxyl, (C₁-C₆)-alkoxy or by a radical of the formulae NR³⁴R³⁵ or —O—CO—R³⁶,

in which

R³⁴ and R³⁵ are identical or different and are hydrogen or (C₁-C₆)-alkyl,

R³⁶ is (C₁-C₆)-alkyl,

or

R⁴ is a radical of the formula —CO—R³⁷,

in which

R³⁷ is a radical of the formulae

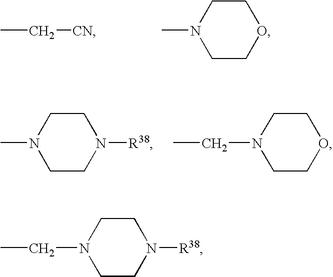

—(CH₂)_{c}—NR³⁹R⁴⁰ or —CH₂—P(O)(OR¹⁴)(OR⁴²),

in which

R³⁵ is hydrogen or (C₁-C₆)-alkyl,

c is either 0 or 1,

R³⁹ and R⁴⁰ are identical or different and are hydrogen or (C₁-C₆)-alkyl, which is optionally substituted by hydroxyl,

R⁴¹ and R⁴² are identical or different and are (C₁-C₆)-alkyl,

or

R⁴ is a 5-membered heterocycle having up to 3 heteroatoms from the series S, N and/or O which is optionally substituted, in the case of an N function also via the latter, a total of up to 3 times, identically or differently, by halogen, trifluoromethyl or by phenyl which may in turn be substituted one or more times by halogen or trifluoromethyl,

and/or is optionally substituted by (C₃-C₆)-cycloalkyl, pyrryl or (C₁-C₁₂)-alkyl which may in turn be substituted by cyano, trifluoromethyl, (C₁-C₆)-alkoxycarbonyl, (C₁-C₆)-alkoxy, amino or by phenyl or nitro-substituted phenyl,

and/or may optionally be substituted by —NR⁴³R^{44,}—NH—CO—CO—R⁴⁵, —NH—CO—R , —NH—CO—CH₂-R⁴⁷, —CO—R or

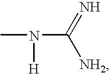

in which

R⁴³ and R⁴⁴ are identical or different and are hydrogen, benzyl (C₁-C₆)-alkyl or phenyl which is optionally substituted by halogen or trifluoromethyl,

R⁴⁵ is (C₁-C₆)-alkoxy,

R⁴⁶ is (C₁-C₆)-alkyl or phenyl,

R⁴⁷ is hydroxyl, (C₁-C₆)-alkoxy or a radical of the formula —O—CO—R⁴⁹, in which

R⁴⁹ is (C₁-C₄)-alkyl,

R⁴⁸ is a radical of the formula —CH₂—CN or phenyl which is optionally substituted by halogen, trifluoromethyl or (C₁-C₆)-alkoxy,

and the salts, tautomers, N-oxides, prodrugs and hydrates thereof, and isomeric forms,

are also suitable for producing medicaments which are employed for the treatment of and/or prophylaxis of coronary heart disease, heart failure, pulmonary hypertension, bladder disorders, prostate hyperplasia, nitrate-induced tolerance, ocular disorders such as glaucoma, for the treatment or prophylaxis of central retinal or posterior cilliary arterial occlusion, central retinal venous occlusion, optic neuropathy such as anterior ischemic optic neuropathy and glaucomatous optic neuropathy, and of macular degeneration, diabetes, especially of diabetic gastroparesis, for the treatment of disorders of the peristalsis of stomach and esophagus, female infertility, premature labor, preeclampsia, alopecia, psoriasis, the renal syndrome, cystic fibrosis, cancer, for improving perception, for improving concentration, for improving leaming and/or memory, in particular if the impairment is a consequence of dementia.

The compounds of the general formula (I) may, depending on the substitution pattern, exist in stereoisomeric forms which either are related as image and mirror image (enantiomers) or which are not related as image and mirror image (diastereomers). The invention relates both to the enantiomers or diastereomers and to respective mixtures thereof. The racemic forms can, just like the diastereomers, be separated into the stereoisomerically pure constituents in a known manner.

Certain compounds of the general formula (I) may moreover exist in tautomeric forms. This is known to the skilled worker, and such compounds are likewise included within the scope of the invention.

Physiologically acceptable, i.e. pharmaceutically suitable salts may be salts of the compounds of the invention with inorganic or organic acids. Preferred salts are those with inorganic acids such as, for example, hydrochloric acid, hydrobromic acid, phosphoric acid or sulfuric acid, or salts. with organic carboxylic or sulfonic acids such as, for example, acetic acid, propionic acid, maleic acid, fumaric acid, malic acid, citric acid, tartaric acid, lactic acid, benzoic acid, or methanesulfonic acid, ethanesulfonic acid, benzenesulfonic acid, toluenesulfonic acid or naphthalenedisulfonic acid.

Pharmaceutically acceptable salts which may also be mentioned are salts with conventional bases such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procaine, dibenzylamine, N-methylmorpholine, dihydroabietylamine or methylpiperidine.

“Hydrates” refer according to the invention to those forms of the compounds of the above general formula (I) which form a molecular compound (solvate) in the solid or liquid state by hydration with water. The water molecules in the hydrates are attached through secondary valences by intramolecular forces, in particular hydrogen bonds. Solid hydrates contain water as so-called water of crystallization in stoichiometric ratios, and the water molecules do not have to be equivalent in terms of their binding state. Examples of hydrates are sesquihydrates, monohydrates, dihydrates or trihydrates. Equally suitable are also the hydrates of salts of the compounds of the invention.

“Prodrugs” refer according to the invention to those forms of the compounds of the above general formula (I) which may themselves be biologically active or inactive but can be converted into the corresponding biologically active form (for example metabolically, solvolytically or in another way).

(C₁-C₁₂)-Alkyl is a straight-chain or branched alkyl radical having 1 to 12 carbon atoms. Examples which may be mentioned are: methyl, ethyl, n-propyl, isopropyl, n-butyl, isobutyl, tert-butyl, n-pentyl and n-hexyl. The corresponding alkyl groups with fewer carbon atoms are derived analogously from this definition, such as, for example, (C₁-C₆)-alkyl and (C₁-C₄)-alkyl. It is generally true that (C₁-C₄)-alkyl is preferred.

(C₃-C₈)-Cycloalkyl is a cyclic alkyl radical having 3 to 8 carbon atoms. Examples which may be mentioned are: cyclopropyl, cyclobutyl, cyclopentyl, cyclohexyl, cycloheptyl or cyclooctyl. The corresponding cycloalkyl groups with fewer carbon atoms are derived analogously from this definition, such as, for example, (C₃-C₅)-cycloalkyl. Cyclopropyl, cyclopentyl and cyclohexyl are preferred.

(C₁-C₆)-Alkoxy is a straight-chain or branched alkoxy radical having 1 to 6 carbon atoms. Examples which may be mentioned are: methoxy, ethoxy, n-propoxy, isopropoxy, n-butoxy, isobutoxy, tert-butoxy, n-pentoxy and n-hexoxy. The corresponding alkoxy groups with fewer carbon atoms are derived analogously from this definition, such as, for example, (C₁-C₆)-alkoxy and (C₁-C₄)-alkoxy. It is generally true that (C₁-C₄)-alkoxy is preferred.

Also derived from this definition is the meaning of the corresponding component of another more complex substituent such as, for example, alkoxycarbonyl.

(C₆-C₁₂)-Aryl is an aromatic radical having 6 to 12 carbon atoms. Examples which may be mentioned are: phenyl and naphthyl.

5- to 6-membered, aromatic or saturated heterocycle having up to 3 heteroatoms from the series S. N and/or 0 is either a heteroaromatic system which is linked via a ring carbon atom of the heteroaromatic system, where appropriate also via a ring nitrogen atom of the heteroaromatic system; examples which may be mentioned are: pyridyl, pyrimidyl, pyridazinyl, pyrazinyl, thienyl, furyl, pyrrolyl, pyrazolyl, imidazolyl, thiazolyl, oxazolyl or isoxazolyl, with preference for pyridyl, pyrimidyl, pyridazinyl, furyl and thienyl, or is a saturated heterocycle which is linked via a ring carbon atom or a ring nitrogen atom, or is a (C₅-C₆)-cycloalkyl radical as defined above; examples which may be mentioned are: tetrahydrofuryl, pyrrolidinyl, piperidinyl, piperazinyl, morpholinyl, thiomorpholinyl, cyclopentyl and cyclohexyl, with preference for piperdinyl, morpholinyl and pyrrolidinyl.

It is preferred to use according to the invention compounds of the general formula (I)

in which

R¹ is (C₁-C₄)-alkyl,

R² is cyclopentyl, cycloheptyl or (C₁-C₁₀)-alkyl,

R³ is (C₁-C₄)-alkyl,

R⁴ is a radical of the formulae

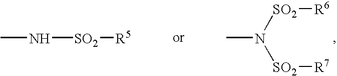

in which

R⁵, R⁶ and R⁷ are identical or different and are vinyl or (C₁-C₄)-alkyl which is optionally substituted up to 3 times, identically or differently, by trifluoromethyl, chlorine, (C₁-C₄)-alkoxy or by radicals of the formulae

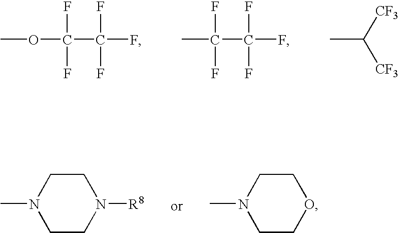

in which

R⁸ is hydrogen, methyl or ethyl,

or

R⁵, R⁶ and/or R⁷ are phenyl which is optionally substituted up to 3 times, identically or differently by halogen, trifluoromethyl, nitro, cyano, carboxyl, (C₁-C₄)-alkyl or (C₁-C₄)-alkoxy,

or

R⁵ is quinolyl or a radical of the formulae

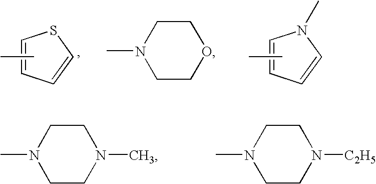

which may optionally be substituted up to twice, identically or differently, by chlorine or (C₁-C₄)-alkyl,

or

R⁵ is a radical of the formulae

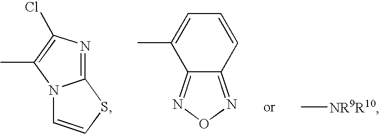

in which

R⁹ and R¹⁰ are identical or different and are hydrogen, (C₁-C₆)-alkyl or phenyl,

or

R⁴ is carboxyl or is a radical of the formulae

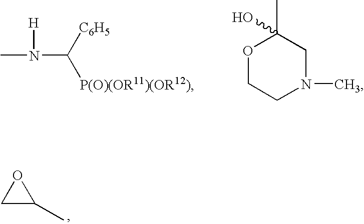

—CO—R¹³ or —O—R¹⁴,

in which

R¹¹ and R¹² are identical or different and are hydrogen or (C₁-C₄)-alkyl,

R¹³ is (C₁-C₄)-alkyl,

R¹⁴ is (C₁-C₄)-alkyl which is optionally substituted up to 3 times, identically or differently, by hydroxyl, phenyl or by a radical of the formula —NR¹⁵R¹⁶,

in which

R¹⁵ and R¹⁶ are identical or different and are hydrogen, phenyl or (C₁-C₄)-alkyl which may in turn be substituted by phenyl,

or

R⁴ is a radical of the formula —NH—CO—NR¹⁷R¹⁸,

in which

R¹⁷ and R¹⁸ are identical or different and are hydrogen or (C₁-C₄)-alkyl which is optionally substituted by hydroxyl or by a radical of the formulae

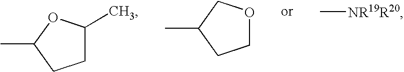

in which

R¹⁹ and R²⁰ are identical or different and are hydrogen, phenyl or (C₁-C₄)-alkyl,

or

R¹⁷ and R¹⁸ form together with the nitrogen atom to which they are bonded a heterocyclic ring of the formulae

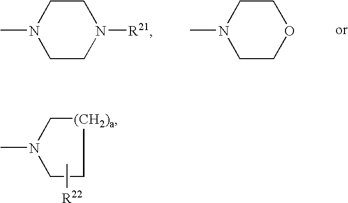

in which

R²¹ is hydrogen or (C₁-C₄)-alkyl,

a is either 1 or 2,

R²² is hydroxyl or (C₁-C₄)-alkyl which is optionally substituted by hydroxyl,

or

R¹⁷ and/or R¹⁸ are phenyl which is optionally substituted by chlorine, trifluoroethyl or by —SCF₃,

or

R¹⁷ is hydrogen, and

R¹⁸ is a radical of the formula —SO₂—R²³,

in which

R²³ is (C₁-C₄)-alkyl or phenyl which is optionally substituted by halogen,

or is a radical of the formulae

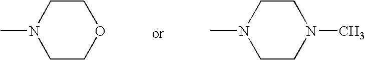

or

R⁴ is a radical of the formula

—NH—CO—R²⁴

in which

R²⁴ is a radical of the formula

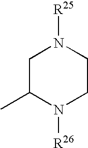

in which

R²⁵ and R²⁶ are identical or different and are hydrogen, (C₁-C₄)-alkyl or (C₁-C₄)-alkoxycarbonyl,

or

R²⁴ is (C₁-C₄)-alkyl which is optionally substituted by phenyl which may in turn be substituted by hydroxyl or (C₁-C₄)-alkoxy, or

(C₁-C₄)-alkyl is optionally substituted by a radical of the formula —(SO₂)_{b}—R²⁷

in which

b is either 0 or 1, and

R²⁷ is a radical of the formulae

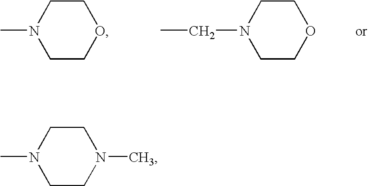

or

R⁴ is (C₁-C₁₁)-alkyl which is optionally substituted up to 3 times, identically or differently, by hydroxyl, azide, phenyl or by radicals of the formulae —NR²⁸R²⁹, —O—CO—R³⁰ or —P(O){O—[(C₁-C₆)-alkyl]}₂,

in which

R²⁸ and R²⁹ are identical or different and are hydrogen, phenyl or (C₁-C₄)-alkyl which is optionally substituted by hydroxyl, (C₁-C₄)-alkoxy or phenyl,

or

R²⁸ and R²⁹ form together with the nitrogen atom to which they are bonded a heterocyclic ring of the formulae

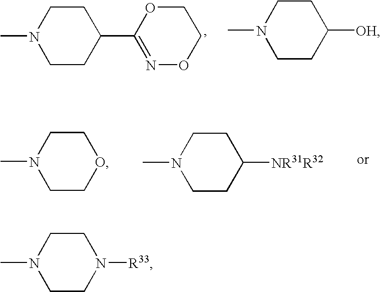

in which

R³¹ and R³² are identical or different and are hydrogen or (C₁-C₄)-alkyl,

R³³ is (C₁-C₄)-alkyl, benzyl, (C₁-C₄)-alkoxycarbonyl, (C₁-C₄)-alkylcarbonyl, carboxyl, pyridyl, pyrimidyl or phenyl which is optionally substituted by (C₁-C₄)-alkoxy,

and

R³⁰ is (C₁-C₆)-alkyl,

or

(C₁-C₁₁)-alkyl is optionally substituted by triazolyl which may in turn be substituted up to twice, identically or differently, by halogen, phenyl, tetrahydrofuranyl, tetrahydropyranyl, (C₁-C₄)-alkoxycarbonyl, aminocarbonyl or by (C₁-C₄)-alkyl, where the latter may optionally be substituted by hydroxyl, (C₁-C₄)-alkoxy or by a radical of the formulae NR³⁴R³⁵ or —O—CO—R³⁶,

in which

R³⁴ and R³⁵ are identical or different and are hydrogen or (C₁-C₄)-alkyl,

R³⁶ is (C₁-C₄)-alkyl,

or

R⁴ is a radical of the formula —CO—R³⁷

in which

R³⁷ is a radical of the formulae

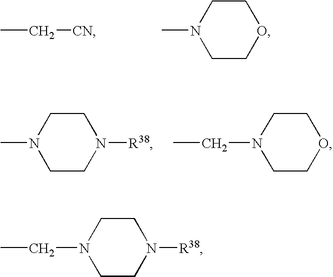

—(CH₂)_{c}—NR³⁹R⁴⁰ or —CH₂—P(O)(R⁴¹ )(OR⁴²),

in which

R³⁸ is hydrogen or (C₁-C₄)-alkyl,

c is either 0 or 1,

R³⁹ and R⁴⁰ are identical or different and are hydrogen or (C₁-C₄)-alkyl which is optionally substituted by hydroxyl,

R⁴¹ and R⁴² are identical or different and are (C₁-C₄)-alkyl,

or

R⁴ is a radical of the formula

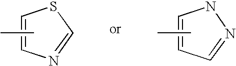

which is optionally substituted, in the case of pyrazole also via the N function, a total of up to 3 times, identically or differently, by chlorine, trifluoromethyl or by phenyl which may in turn be substituted one or more times by chlorine or trifluoromethyl,

and/or is optionally substituted by cyclopentyl, cyclohexyl, pyrryl or (C₁-C₁₂)-alkyl which may in turn be substituted by cyano, trifluoromethyl, (C₁-C₄)-alkoxycarbonyl, (C₁-C₄)-alkoxy, amino or by phenyl or nitro-substituted phenyl,

and/or may also be substituted by —NR⁴³R⁴⁴, —NH—CO—CO—R⁴⁵, —NH—CO—R⁴⁶,

—NH—CO—CH₂—R⁴⁷, —CO—R⁴⁸ or

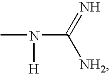

in which

R⁴³ and R⁴⁴ are identical or different and are hydrogen, benzyl, (C₁-C₄)-alkyl or phenyl which is optionally substituted by halogen or trifluoromethyl,

R⁴⁵ is (C₁-C₅)-alkoxy,

R⁴⁶ is (C₁-C₅)-alkyl or phenyl,

R⁴⁷ is hydroxyl, (C₁-C₄)-alkoxy or a radical of the formula —O—CO—R⁴⁹,

in which

R⁴⁹ is (C₁-C₃)-alkyl,

R⁴⁸ is a radical of the formula —CH₂—CN or phenyl which is optionally substituted by chlorine, trifluoromethyl or (C₁-C₄)-alkoxy,

and the N-oxides and/or tautomers thereof, and the pharmaceutically suitable salts, hydrates and prodrugs thereof.

Particular preference is given to the compounds of the invention of the general formula (I)

in which

R¹ is (C₁-C₄)-alkyl,

R² is cyclopentyl, cyclohexyl, cycloheptyl or (C₁-C₁₀)-alkyl,

R³ is (C₁-C₄)-alkyl,

R⁴ is a radical of the formulae

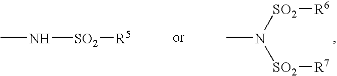

in which

R⁵, R⁶ and R⁷ are identical or different and are vinyl or (C₁-C₄)-alkyl which is optionally substituted up to 3 times, identically or differently, by trifluoromethyl, chlorine, (C₁-C₄)-alkoxy or by radicals of the formulae

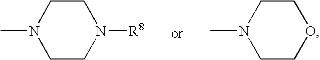

in which

R⁸ is hydrogen, methyl or ethyl,

or

R⁵ R⁶ and/or R⁷ are phenyl which is optionally substituted up to 3 times, identically or differently, by halogen, cyano, (C₁-C₄)-alkyl or (C₁-C₄)-alkoxy,

or

R⁵ is a radical of the formulae

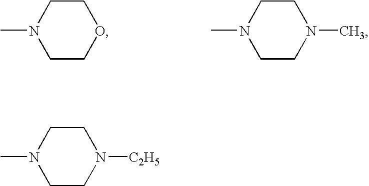

which may optionally be substituted up to twice, identically or differently, by chlorine or (C₁-C₄)-alkyl,

or

R⁵ is a radical of the formula —NR⁹R¹⁰,

in which

R⁹ and R¹⁰ are identical or different and are hydrogen, (C₁-C₄)-alkyl or phenyl,

or

R⁴ is carboxyl or is a radical of the formulae

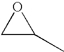

or

—CO—R¹³ or —O—R¹⁴,

in which

R¹³ is (C₁-C₄)-alkyl,

R¹⁴ is (C₁-C₄)-alkyl which is optionally substituted up to 3 times, identically or differently, by hydroxyl or by a radical of the formula —NR¹⁵R¹⁶

in which

R¹⁵ and R¹⁶ are identical or different and are hydrogen or (C₁-C₄)-alkyl which in turn may be substituted by phenyl,

or

R⁴ is a radical of the formula —NH—CO—NR¹⁷R¹⁸,

in which

R¹⁷ and R¹⁸ are identical or different and are hydrogen or (C₁-C₄)-alkyl which is optionally substituted by hydroxyl,

or

R¹⁷ and R¹⁸ form together with the nitrogen atom to which they are bonded a heterocyclic ring of the formulae

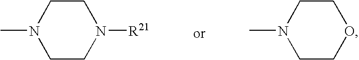

in which

R²¹ is hydrogen or (C₁-C₄)-alkyl,

or

R¹⁷ and/or R¹⁸ are phenyl which is optionally substituted by chlorine, trifluoroethyl or by —SCF₃,

or

R¹⁷ is hydrogen, and

R¹⁸ is a radical of the formula —SO₂—R²³,

in which

R²³ is (C₁-C₄)-alkyl or phenyl which is optionally substituted by halogen,

or is a radical of the formulae

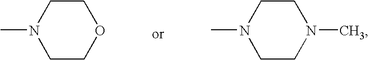

or

R⁴ is a radical of the formula

—NH—CO—R²⁴,

in which

R²⁴ is (C₁-C₄)-alkyl which is optionally substituted by phenyl which in turn may optionally be substituted by hydroxyl or (C₁-C₄)-alkoxy, or

(C₁-C₄)-alkyl is optionally substituted by a radical of the formula —(SO₂)_{b}—R²⁷,

in which

b is either 0 or 1, and

R²⁷ is a radical of the formulae

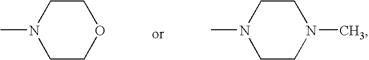

or

R⁴ is (C₁-C₆)-alkyl which is optionally substituted up to 3 times, identically or differently, by hydroxyl, phenyl or by radicals of the formulae —NR²⁸R²⁹ or —O—CO—R³⁰,

in which

R²⁸ and R²⁹ are identical or different, are hydrogen, phenyl or (C₁-C₄)-alkyl which is optionally substituted by hydroxyl, (C₁-C₄)-alkoxy or phenyl,

or

R²⁸ and R²⁹ form together with the nitrogen atom to which they are bonded a heterocyclic ring of the formulae

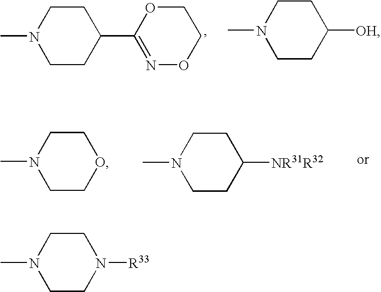

in which

R³¹ and R³² are identical or different and are hydrogen or (C₁-C₄)-alkyl,

R³³ is (C₁-C₄)-alkyl, benzyl, (C₁-C₄)-alkoxycarbonyl, (C₁-C₄)-alkylcarbonyl, carboxyl, pyridyl, pyrimidyl or phenyl which is optionally substituted by (C₁-C₄)-alkoxy,

and

R³⁰ is (C₁-C₆)-alkyl,

or

(C₁-C₆)-alkyl is optionally substituted by triazolyl which may in turn be substituted up to twice, identically or differently, by (C₁-C₄)-alkyl, where the latter may optionally be substituted by hydroxyl or (C₁-C₄)-alkoxy,

in which

or

R⁴ is a radical of the formula —CO—R³⁷,

in which

R³⁷ is a radical of the formulae

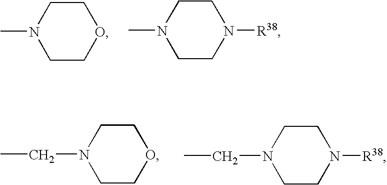

or —(CH₂)_{c}—NR³⁹R⁴⁰,

in which

R³⁸ is hydrogen or (C₁-C₄)-alkyl,

c is either 0or 1,

R³⁹ and R ⁴⁰ are identical or different and are hydrogen or (C₁-C₄)-alkyl which is optionally substituted by hydroxyl,

or

R ⁴ is a radical of the formula

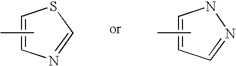

which is optionally substituted, in the case of pyrazole also via the N function, a total of up to 3 times, identically or differently, by trifluoromethyl or by phenyl which may in turn be substituted one or more times by chlorine or trifluoromethyl, and/or is optionally substituted by cyclopentyl, cyclohexyl or by (C₁-C₆)-alkyl which may in turn be substituted by (C₁-C₄)-alkoxy, amino or by phenyl,

and/or may optionally be substituted by —NR⁴³R⁴⁴, —NH—CO—R⁴⁶, —NH—CO—CH₂—R⁴⁷ or —CO—R⁴⁸,

in which

R⁴³ and R⁴⁴ are identical or different and are hydrogen, benzyl, (C₁-C₄)-alkyl or phenyl which is optionally substituted by halogen or trifluoromethyl,

R⁴⁶ is (C₁-C₄)-alkyl or phenyl,

R⁴⁷ is hydroxyl or (C₁-C₄)-alkoxy,

R⁴⁸ is phenyl which is optionally substituted by chlorine, trifluoromethyl or (C₁-C₄)-alkoxy,

and the N-oxides and/or tautomers thereof, and the pharmaceutically acceptable salts, hydrates and prodrugs thereof.

Very particular preference is given to the compounds of the invention having the following structures:

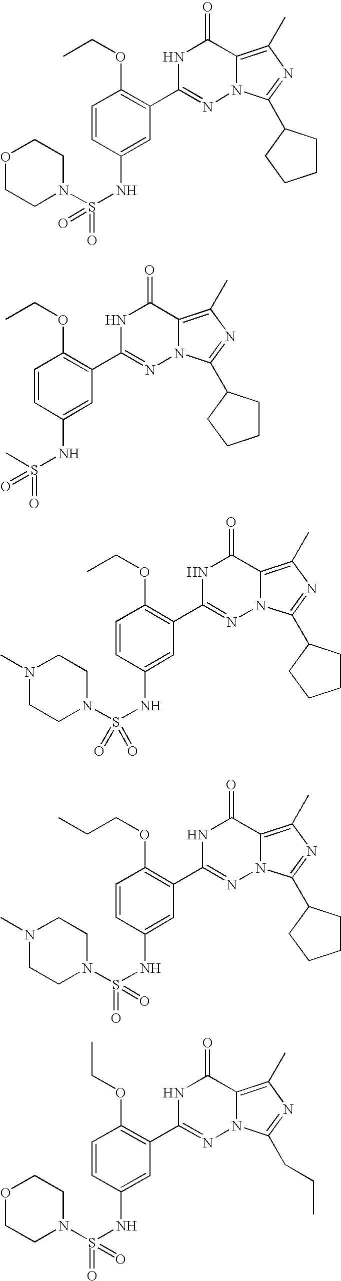

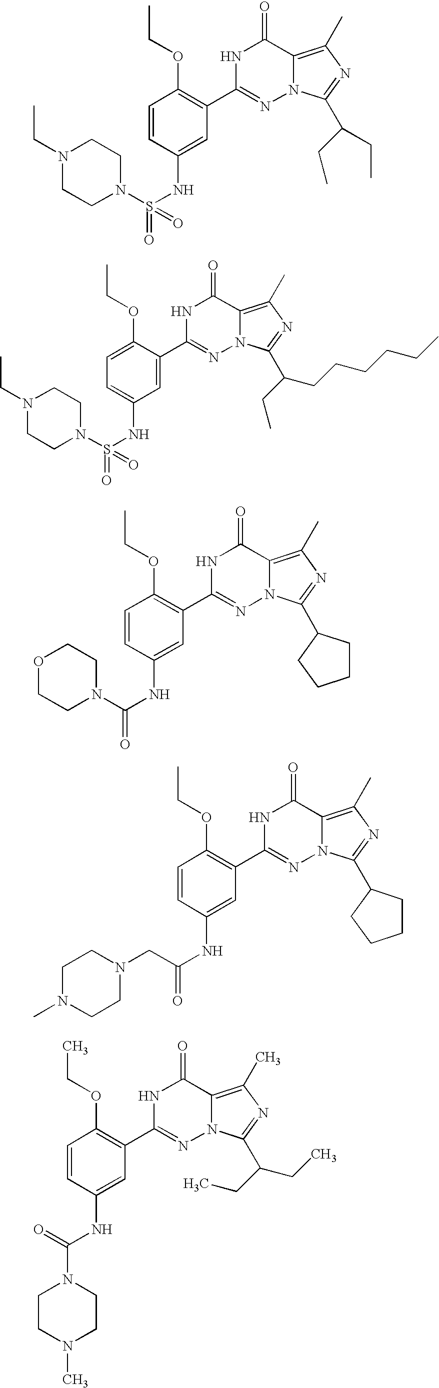

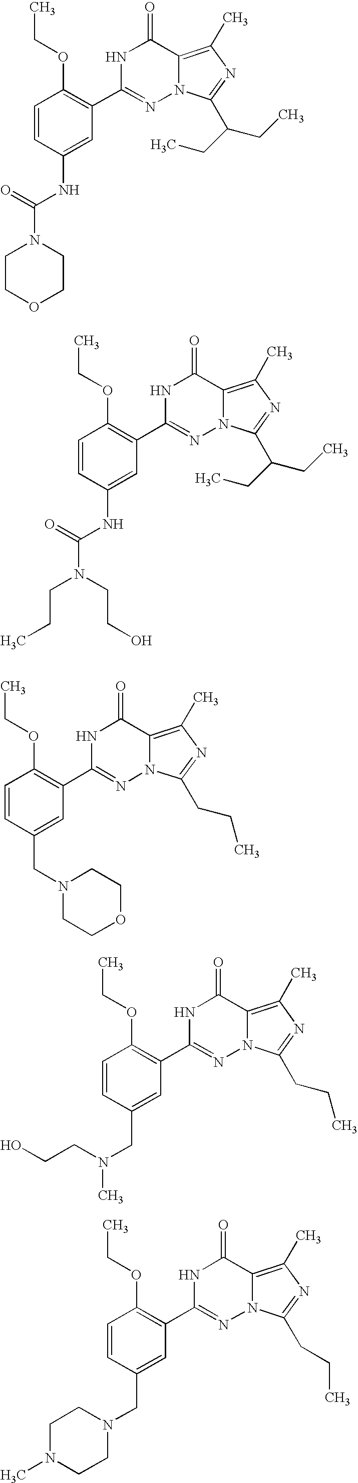

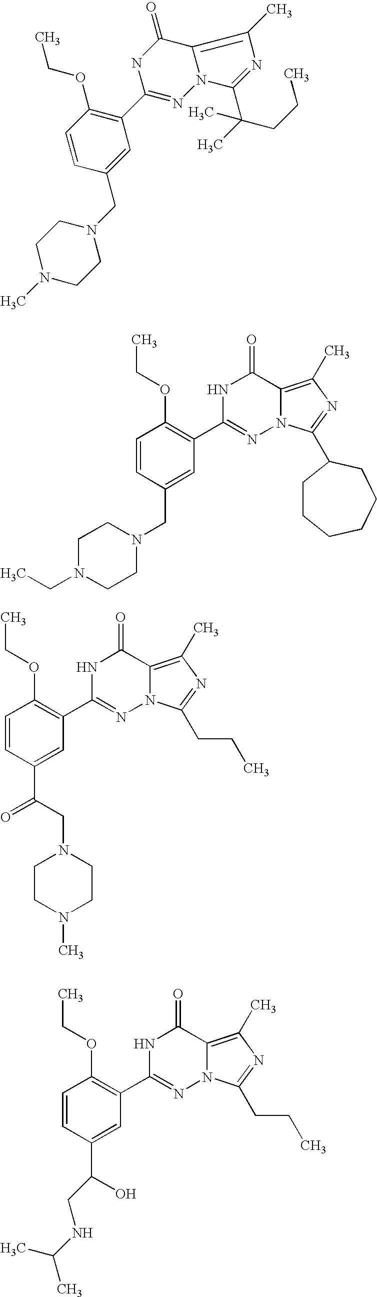

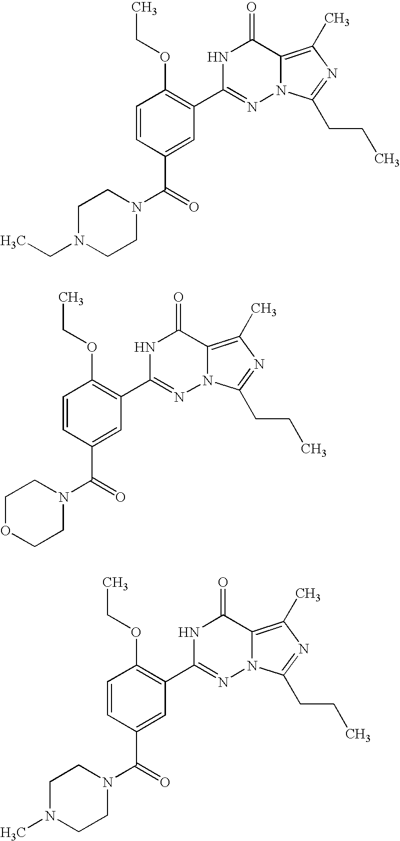

and the tautomers and/or N-oxides thereof, and the pharmaceutically acceptable salts, hydrates and prodrugs thereof.

The compounds used according to the invention and their preparation are described in WO-A 01/64677. Express reference is made to the disclosure of WO-A 01/64677.

The compounds of the general formula (I) which are used according to the invention are suitable for the prophylaxis and/or treatment of disorders in which an increase in the cGMP concentration is therapeutic, i.e. disorders associated with cGMP-regulated processes (usually referred to simply as ‘cGMP-related diseases’). They inhibit either one or a plurality of the cGMP-metabolizing phosphodiesterases (PDE I, PDE II and PDE V). This leads to an increase in cGMP. Differential expression of the phosphodiesterases in different cells, tissues and organs, as well as the differential subcellular localization of these enzymes, make it possible in conjunction with the selective inhibitors of the invention to address selectively the various cGMP-regulated processes.

The relaxant effect on smooth muscle makes them suitable for the treatment of disorders in which an improvement and/or cure of a pathological condition can be achieved by improving the microcirculation of a tissue which comprises a cGMP-metabolizing phosphodiesterase.

The present invention relates to the use of imidazotriazinones for producing a medicament for the treatment and/or prophylaxis of coronary heart disease, heart failure, pulmonary hypertension, bladder disorders, prostate hyperplasia, nitrate-induced tolerance, ocular disorders such as glaucoma, for the treatment or prophylaxis of central retinal or posterior cilliary arterial occlusion, central retinal venous occlusion, optic neuropathy such as anterior ischemic optic neuropathy and glaucomatous optic neuropathy, and of macular degeneration, diabetes, especially of diabetic gastroparesis, for the treatment of disorders of the peristalsis of stomach and esophagus, female infertility, premature labor, preeclampsia, alopecia, psoriasis, the renal syndrome, cystic fibrosis, cancer, for improving perception, for improving concentration, for improving learning and/or memory, especially if the impairment is a consequence of dementia.

In addition, the compounds of the invention enhance the effect of substances such as, for example, EDRF (endothelium derived relaxing factor), ANP (atrial natriuretic peptide), of nitrate vasodilators and all other substances which increase the cGMP concentration in a way different from phosphodiesterase inhibitors.

### Activity of Phosphordiesterases (PDEs)

The cGMP-stimulable PDE II, the cGMP-inhibitable PDE III and the cAMP-specific PDE IV were isolated either from myocardium of porcine or bovine heart. The Ca²⁺ calmodulin-stimulable PDE I was isolated from porcine aorta, porcine brain or preferably from bovine aorta. The cGMP-specific PDE V was obtained from porcine small intestine, porcine aorta, human blood platelets and preferably from bovine aorta. Purification took place by anion exchange chromatography on MonoQ® Pharmacia essentially by the method of M. Hoey and Miles D. Houslay, Biochemical Pharmacology, Vol. 40, 193-202 (1990) and C. Lugman et al. Biochemical Pharmacology Vol. 35 1743-1751 (1986).

The enzymic activity is determined in an assay mixture of 100 μl in 20 mM Tris/HCl buffer pH 7.5 which contains 5 mM MgCl₂, 0.1 mg/ml bovine serum albumin and either 800 Bq of ³HcAMP or ³HcGMP. The final concentration of the appropriate nucleotides is 10-6 mol/l. The reaction is started by adding the enzyme, and the amount of enzyme is such that about 50% of the substrate is converted during the incubation time of 30 min. In order to assay the cGMP-stimulable PDE II, ³HcAMP is used as substrate, and 10-6 moll unlabeled cGMP is added to the mixture. In order to assay the Ca²+-calmodulin-dependent PDE I, 1 μM CaCl₂ and 0.1 μM calmodulin are also added to the reaction mixture. The reaction is stopped by adding 100 μl of acetonitrile containing 1 mM cAMP and 1 mM AMP. 100 μl of the reaction mixture are separated by HPLC, and the cleavage products are determined quantitatively online using a flow scintillation counter. The substance concentration at which the reaction rate is reduced by 50% is measured. Additionally used for assaying was the phosphodiesterase [³H] cAMP-SPA enzyme assay and the phosphodiesterase [³H] cGMP-SPA enzyme assay from Amersham Life Science. The assay was carried out according to the test protocol indicated by the manufacturer. The PDE II activity was determined using the [³H] cAMP SPA assay with addition of 10⁻⁶ cGMP to the reaction mixture to activate the enzyme. For the PDE I measurement, 10⁻⁷ M calmodulin and 1 μM CaCl₂ were added to the reaction mixture. PDE V was measured using the [³H] cGMP SPA assay.

### Object Recognition Test

The object recognition test is a memory test. It measures the ability of rats (and mice) to distinguish between familiar and unfamiliar objects.

The test was carried out as described: Blokland et al, NeuroReport 1998, 9, 4205; Ennaceur et al, Behav. Brain Res. 1988, 31, 47-59; Ennaceur et al, Psychopharmacology 1992, 109, 321-330; Prickaerts et al, Eur. J. Pharmnacol. 1997, 337, 125-136.

Inhibition of one or more phosphodiesterases of this type always leads to an increase in the cGMP concentration. The compounds are therefore of interest for all therapies in which an increase in the cGMP concentration can be assumed to be therapeutic.

The investigation of the cardiovascular effects was carried out on normotensive and on SH rats and on dogs. The substances were administered intravenously or orally.

The novel active ingredients and their physiologically acceptable salts (e.g. hydrochlorides, maleates or lactates) can be converted in a known manner into conventional formulations such as tablets, coated tablets, pills, granules, aerosols, syrups, emulsions, suspensions and solutions, using inert, nontoxic, pharmaceutically suitable carriers or solvents. In these, the therapeutically active compounds should be present in each case in a concentration of about 0.5 to 90% by weight of the complete mixture, i.e. in amounts which are sufficient to achieve the stated dosage range.

The formulations are produced for example by extending the active ingredients with solvents and/or carriers, where appropriate with use of emulsifiers and/or dispersants, it being possible, for example when water is used as diluent, where appropriate to use organic solvents as auxiliary solvents.

Administration takes place in a conventional way, preferably orally, transdermally or parenterally, i.e. perlingually, sublingually, conjunctivally, optically, buccally, intravenously, nasally, rectally, by inhalation or as implant.

For use in humans, generally dosages of from 0.001 to 50 mg/kg, preferably 0.01 mg/kg-20 mg/kg, are administered on oral administration. A dosage appropriate for parenteral administration such as, for example, nasally, buccally or by inhalation via mucous membrane is 0.001 mg/kg-0.5 mg/kg.

It may nevertheless be necessary where appropriate to deviate from the amounts mentioned, in particular as a function of the body weight or of the nature of the administration route, of the individual response to the medicament, of the nature of its formulation and the time or interval over which administration takes place. Thus, in some cases it may suffice to make do with less than the aforementioned minimum amount, whereas in other cases the stated upper limit must be exceeded. Where larger amounts are administered, it may be advisable to divide these into a plurality of single doses over the day.

The compounds of the invention are also suitable for use in veterinary medicine. For uses in veterinary medicine, the compounds or their nontoxic salts can be administered in a suitable formulation complying with general veterinary practices. The veterinarian is able to establish the mode of use and the dosage according to the species of the animal to be treated.

The present invention is illustrated by the following examples which are, however, not intended to restrict the invention in any way.

1. A method for the prophylaxis and/or treatment of disorders associated with cGMP-regulated processes or cGMP-related diseases comprising administering to a subject an effective amount of a compound of formula (I) 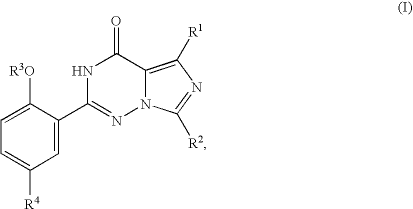
in which
R¹ is (C₁-C₆)-alkyl,
R² is (C₃-C₈)-cycloalkyl or (C₁-C₁₂)-alkyl,
R³ is (C₁-C₆)-alkyl,
R⁴ is a radical of the formulae 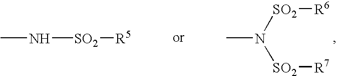
in which
R⁵, R⁶ and R⁷ are identical or different and are vinyl or (C₁-C₆)-alkyl which is optionally substituted up to 3 times, identically or differently, by trifluoromethyl, halogen, (C₁-C₆)-alkoxy or by radicals of the formulae 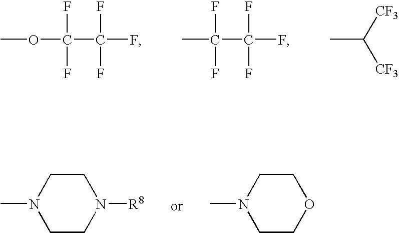
in which
R⁸ is hydrogen or (C₁-C₄)-alkyl, or
R⁵, R⁶ and/or R⁷ are (C₆-CI₂)-aryl which is optionally substituted up to 3 times, identically or differently, by halogen, trifluoromethyl, nitro, cyano, carboxyl, (C₁-C₆)-alkyl or (C₁-C₆)-alkoxy, or
R⁵ is quinolyl or a 5- to 6-membered, aromatic or saturated heterocycle having up to 3 heteroatoms from the series S, N and/or O, which may optionally be substituted, in the case of an N function also via the latter, up to 3 times, identically or differently, by halogen or (C₁-C₆)-alkyl, or
R⁵ is a radical of the formulae 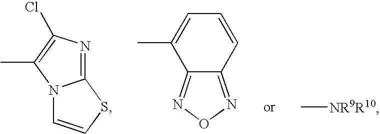
in which
R⁹ and R¹⁰ are identical or different and are hydrogen, (C₁-C₆)-alkyl or phenyl, or
R⁴ is carboxyl or is a radical of the formulae 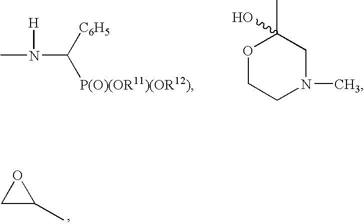
—CO—R¹³ or —O—R¹⁴,
in which
R¹¹ and R¹² are identical or different and are hydrogen or (C₁-C₄)-alkyl,
R¹³ is (C₁-C₆)-alkyl,
R¹⁴ is (C₁-C₆)-alkyl which is optionally substituted up to 3 times, identically or differently, by hydroxyl, phenyl or by a radical of the formula —NR¹⁵R¹⁶,
in which
R¹⁵ and R¹⁶ are identical or different and are hydrogen, phenyl or (C₁-C₄)-alkyl which in turn may be substituted by phenyl, or
R⁴ is a radical of the formula —NH—CO—NR¹⁷R¹⁸,
in which
R¹⁷ and R¹⁸ are identical or different and are hydrogen or (C₁-C₆)-alkyl which is optionally substituted by hydroxyl or by a radical of the formulae 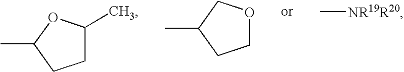
in which
R¹⁹ and R²⁰ are identical or different and are hydrogen, phenyl or (C₁-C₆)-alkyl, or
R¹⁷ and R¹⁸ form together with the nitrogen atom to which they are bonded a heterocyclic ring of the formulae 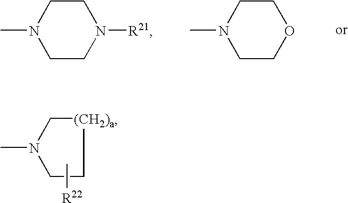
in which
R²¹ is hydrogen or (C₁-C₆)-alkyl,
a is either 1 or 2,
R²² is hydroxyl or (C₁-C₆)-alkyl which is optionally substituted by hydroxyl, or
R¹⁷ and/or R¹⁸ are (C₆-C₁₂)-aryl which is optionally substituted by halogen, trifluoroethyl or by —SCF₃, or
R¹⁷ is hydrogen and
R¹⁸ is a radical of the formula —SO₂—R
in which
R²³ is (C₁-C₆)-alkyl or (C₆-C₁₂)-aryl which is optionally substituted by halogen, or is a radical of the formulae 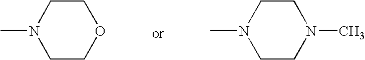
or
R⁴ is a radical of the formula
—NH—CO—R²⁴,
in which
R²⁴ is a radical of the formula 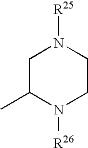
in which
R²⁵ and R²⁶ are identical or different and are hydrogen, (C₁-C₆)-alkyl or (C₁-C₆)-alkoxycarbonyl, or
R²⁴ is (C₁-C₆)-alkyl which is optionally substituted by (C₆-C₁₂)-aryl which in turn may be substituted by hydroxyl or (C₁-C₆)-alkoxy or
(C₁-C₆)-alkyl is optionally substituted by a radical of the formula —(SO₂)_{b}—R²⁷, in which
b is either 0 or 1, and
R²⁷ is a radical of the formulae 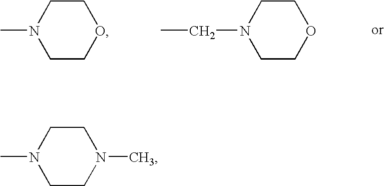
or
R⁴ is (C₁-C₁₂)-alkyl which is optionally substituted up to 3 times, identically or differently, by hydroxyl, azide, phenyl or by radicals of the formulae —NR²⁸R²⁹, —O—CO—R³⁰ or —P(O) {O—[(C₁-C₆)-alkyl]}₂,
in which
R²⁸ and R²⁹ are identical or different, are hydrogen, phenyl or (C₁-C₆)-alkyl which is optionally substituted by hydroxyl, (C₁-C₆)-alkoxy or phenyl, or
R²⁸ and R²⁹ form together with the nitrogen atom to which they are bonded a heterocyclic ring of the formulae 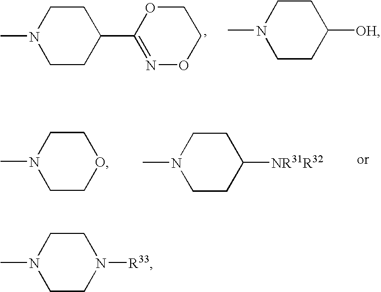
in which
R³¹ and R³² are identical or different and are hydrogen or (C₁-C₆)-alkyl,
R³³ is (C₁-C₆)-alkyl, benzyl, (C₁-C₆)-alkoxycarbonyl, (C₁-C₆)-alkylcarbonyl, carboxyl, pyridyl, pyrimidyl or phenyl which is optionally substituted by (C₁-C₆)-alkoxy, and
R³⁰ is (C₁-C₆)-alkyl, or
(C₁-C₁₂)-alkyl is optionally substituted by triazolyl which may in turn be substituted up to twice, identically or differently, by halogen, phenyl, tetrahydrofuranyl, tetrahydropyranyl, (C₁-C₆)-alkoxycarbonyl, aminocarbonyl or by (C₁-C₆)-alkyl, where the latter can optionally be substituted by hydroxyl, (C₁-C₆)-alkoxy or by a radical of the formulae NR³⁵R³⁵ or —O—CO—R³⁶, in which
R³⁴ and R³⁵ are identical or different and are hydrogen or (C₁-C₆)-alkyl,
R³⁶ is (C₁-C₆)-alkyl, or
R⁴ is a radical of the formula CO—R³⁷, in which
R³⁷ is a radical of the formulae 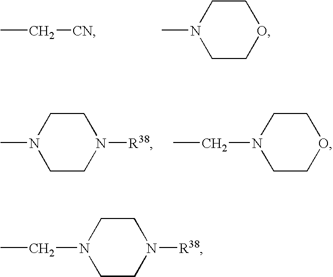
—(CH₂)_{c}—NR³⁹R⁴⁰ or —CH₂—P(O)(OR⁴¹)(OR⁴²),
in which
R³⁸ is hydrogen or (C₁-C₆)-alkyl,
c is either 0 or 1,
R³⁹ and R⁴⁰ are identical or different and are hydrogen or (C₁-C₆)-alkyl, which is optionally substituted by hydroxyl,
R⁴¹ and R⁴² are identical or different and are (C₁-C₆)-alkyl, or
R⁴ is a 5-membered heterocycle having up to 3 heteroatoms from the series S, N and/or O which is optionally substituted, in the case of an N function also via the latter, a total of up to 3 times, identically or differently, by halogen, trifluoromethyl or by phenyl which may in turn be substituted one or more times by halogen or trifluoromethyl,
and/or is optionally substituted by (C₃-C₆)-cycloalkyl, pyrryl or (C₁-CI₂)-alkyl which may in turn be substituted by cyano, trifluoromethyl, (C₁-C₆)-alkoxycarbonyl, (C₁-C₆)-alkoxy, amino or by phenyl or nitro-substituted phenyl,
and/or may optionally be substituted by —NR⁴³R⁴⁴, —NH—CO—CO—R⁴⁵, —NH—CO—R⁴⁶, —NH—CO—CH₂—R⁴⁷, —CO—R⁴⁸ or 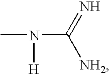
in which
R⁴³ and R⁴⁴ are identical or different and are hydrogen, benzyl (C₁-C₆)-alkyl or phenyl which is optionally substituted by halogen or trifluoromethyl,
R⁴⁵ is (C₁-C₆)-alkoxy,
R⁴⁶ is (C₁-C₆)-alkyl or phenyl,
R⁴⁷ is hydroxyl, (C₁-C₆)-alkoxy or a radical of the formula —O—CO—R⁴⁹, in which
R⁴⁹ is (C₁-C₄)-alkyl,
R⁴⁸ is a radical of the formula —CH₂—CN or phenyl which is optionally substituted by halogen, trifluoromethyl or (C₁-C₆)-alkoxy,
and the salts, tautomers, N-oxides, prodrugs and hydrates thereof, and isomeric forms,

2. The method as claimed in claim 1, wherein the compounds of formula (I)
in which
R¹ is (C₁-C₄)-alkyl,
R² is cyclopentyl, cycloheptyl or (C₁-C₁₀)-alkyl,
R³ is (C₁-C₄)-alkyl,
R⁴ is a radical of the formulae 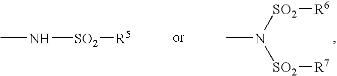
in which
R⁵, R⁶ and R⁷ are identical or different and are vinyl or (C₁-C₄)-alkyl which is optionally substituted up to 3 times, identically or differently, by trifluoromethyl, chlorine, (C₁-C₄)-alkoxy or by radicals of the formulae 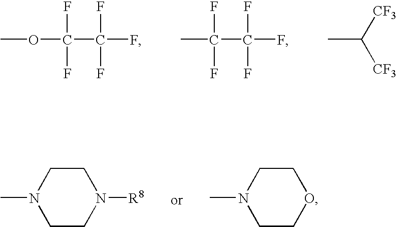
in which
R⁸ is hydrogen, methyl or ethyl, or
R⁵, R⁶ and/or R⁷ are phenyl which is optionally substituted up to 3 times, identically or differently by halogen, trifluoromethyl, nitro, cyano, carboxyl, (C₁-C₄)-alkyl or (C₁-C₄)-alkoxy, or
R⁵ is quinolyl or a radical of the formulae 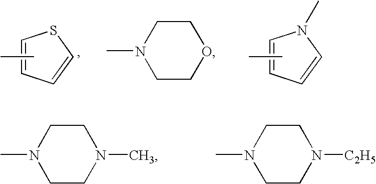
which may optionally be substituted up to twice, identically or differently, by chlorine or (C₁-C₄)-alkyl, or
R⁵ is a radical of the formulae 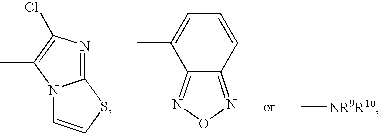
in which
R⁹ and R¹⁰ are identical or different and are hydrogen, (C₁-C₆)-alkyl or phenyl, or
R⁴ is carboxyl or is a radical of the formulae 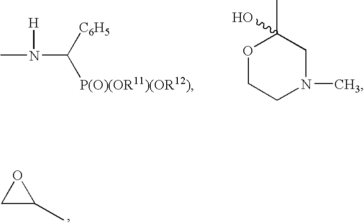
—CO—R¹³ or —O—R¹⁴,
in which
R¹¹ and R¹² are identical or different and are hydrogen or (C₁-C₄)-alkyl,
R¹³ is (C₁-C₄)-alkyl,
R¹⁴ is (C₁-C₄)-alkyl which is optionally substituted up to 3 times, identically or differently, by hydroxyl, phenyl or by a radical of the formula —NR¹⁵R¹⁶,
in which
R¹⁵ and R¹⁶ are identical or different and are hydrogen, phenyl or (C₁-C₄)-alkyl which may in turn be substituted by phenyl, or
R⁴ is a radical of the formula —NH—CO—NR¹⁷R¹⁸,
in which
R¹⁷ and R¹⁸ are identical or different and are hydrogen or (C₁-C₄)-alkyl which is optionally substituted by hydroxyl or by a radical of the formulae 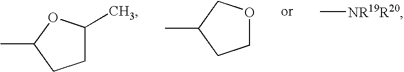
in which
R¹⁹ and R²⁰ are identical or different and are hydrogen, phenyl or (C₁-C₄)-alkyl, or
R¹⁷ and R¹⁸ form together with the nitrogen atom to which they are bonded a heterocyclic ring of the formulae 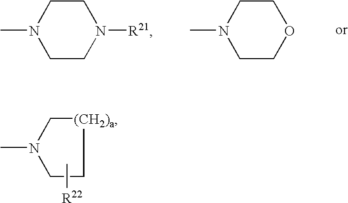
in which
R²¹ is hydrogen or (C₁-C₄)-alkyl,
a is either 1 or 2,
R²² is hydroxyl or (C₁-C₄)-alkyl which is optionally substituted by hydroxyl, or
R¹⁷ and/or R¹⁸ are phenyl which is optionally substituted by chlorine, trifluoroethyl or by —SCF₃, or
R¹⁷ is hydrogen, and
R¹⁸ is a radical of the formula —SO₂—R²³,
in which
R²³ is (C₁-C₄)-alkyl or phenyl which is optionally substituted by halogen, or is a radical of the formulae 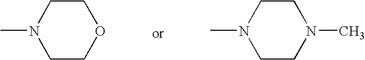
or
R⁴ is a radical of the formula
—NH—CO—R²⁴
in which
R²⁴ is a radical of the formula 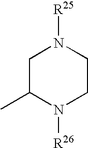
in which
R²⁵ and R²⁶ are identical or different and are hydrogen, (C₁-C₄)-alkyl or (C₁-C₄)-alkoxycarbonyl, or
R²⁴ is (C₁-C₄)-alkyl which is optionally substituted by phenyl which may in turn be substituted by hydroxyl or (C₁-C₄)-alkoxy, or
(C₁-C₄)-alkyl is optionally substituted by a radical of the formula -(SO₂)_{b}—R²⁷ in which
b is either 0 or 1, and
R²⁷ is a radical of the formulae 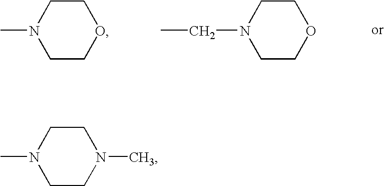
or
R⁴ is (C₁-C₁₁)-alkyl which is optionally substituted up to 3 times, identically or differently, by hydroxyl, azide, phenyl or by radicals of the formulae —NR²⁸R²⁹, —O—CO—R³⁰ or —P(O){O—[(C₁-C₆)-alkyl]}₂,
in which
R²⁸ and R²⁹ are identical or different and are hydrogen, phenyl or (C₁-C₄)-alkyl which is optionally substituted by hydroxyl, (C₁-C₄)-alkoxy or phenyl, or
R²⁸ and R²⁹ form together with the nitrogen atom to which they are bonded a heterocyclic ring of the formulae 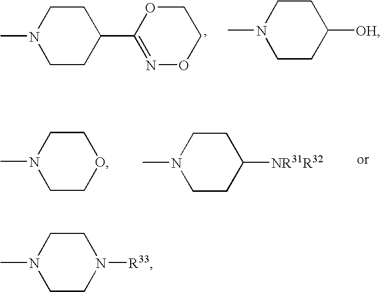
in which
R³¹ and R³² are identical or different and are hydrogen or (C₁-C₄)-alkyl,
R³³ is (C₁-C₄)-alkyl, benzyl, (C₁-C₄)-alkoxycarbonyl, (C₁-C₄)-alkylcarbonyl, carboxyl, pyridyl, pyrimidyl or phenyl which is optionally substituted by (C₁-C₄)-alkoxy, and
R³⁰ is (C₁-C₆)-alkyl, or
(C₁-C₁₁)-alkyl is optionally substituted by triazolyl which may in turn be substituted up to twice, identically or differently, by halogen, phenyl, tetrahydrofuranyl, tetrahydropyranyl, (C₁-C₄)-alkoxycarbonyl, aminocarbonyl or by (C₁-C₄)-alkyl, where the latter may optionally be substituted by hydroxyl, (C₁-C₄)-alkoxy or by a radical of the formulae NR³⁴R³⁵ or —O—CO—R³⁶,
in which
R³⁴ and R³⁵ are identical or different and are hydrogen or (C₁-C₄)-alkyl,
R³⁶ is (C₁-C₄)-alkyl, or
R⁴ is a radical of the formula —CO—R³⁷
in which
R³⁷ is a radical of the formulae 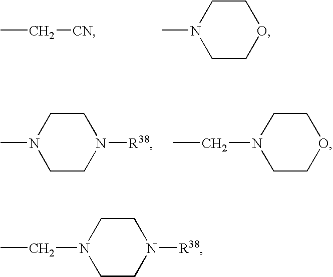
—(CH₂)_{c}—NR³⁹R⁴⁰ or —CH₂—P(O)(OR⁴¹)(OR⁴²),
in which
R³⁸ is hydrogen or (C₁-C₄)-alkyl,
c is either 0 or 1,
R³⁹ and R⁴⁰ are identical or different and are hydrogen or (C₁-C₄)-alkyl which is optionally substituted by hydroxyl,
R⁴′ and R⁴² are identical or different and are (C₁-C₄)-alkyl, or
R⁴ is a radical of the formula 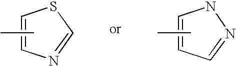
which is optionally substituted, in the case of pyrazole also via the N function, a total of up to 3 times, identically or differently, by chlorine, trifluoromethyl or by phenyl which may in turn be substituted one or more times by chlorine or trifluoromethyl,
and/or is optionally substituted by cyclopentyl, cyclohexyl, pyrryl or (C₁-CI₂)-alkyl which may in turn be substituted by cyano, trifluoromethyl, (C₁-C₄)-alkoxycarbonyl, (C₁-C₄)-alkoxy, amino or by phenyl or nitro-substituted phenyl,
and/or may also be substituted by —NR⁴³R⁴⁴, —NH—CO—CO—R⁴⁵, —NH—CO—R⁴⁶, 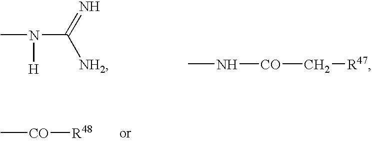
in which
R⁴³ and R⁴⁴ are identical or different and are hydrogen, benzyl, (C₁-C₄)-alkyl or phenyl which is optionally substituted by halogen or trifluoromethyl,
R⁴⁵ is (C₁-C₅)-alkoxy,
R⁴⁶ is (C₁-C₅)-alkyl or phenyl,
R⁴⁷ is hydroxyl, (C₁-C₄)-alkoxy or a radical of the formula —O—CO—R⁴⁹, in which
R⁴⁹ is (C₁-C₃)-alkyl,
R⁴⁸ is a radical of the formula —CH₂—CN or phenyl which is optionally substituted by chlorine, trifluoromethyl or (C₁-C₄)-alkoxy,
and the tautomers thereof, and the pharmaceutically acceptable salts, hydrates and prodrugs thereof.

3. The method as claimed in claim 1, wherein the compound of the formula (I) in which
R¹ is (C₁-C₄)-alkyl,
R² is cyclopentyl, cyclohexyl, cycloheptyl or (C₁-C₁₀)-alkyl,
R³ is (C₁-C₄)-alkyl,
R⁴ is a radical of the formulae 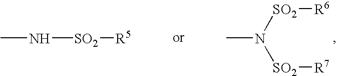
in which
R⁵, R⁶ and R⁷ are identical or different and are vinyl or (C₁-C₄)-alkyl which is optionally substituted up to 3 times, identically or differently, by trifluoromethyl, chlorine, (C₁-C₄)-alkoxy or by radicals of the formulae 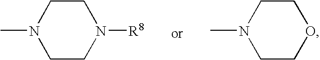
in which
R⁸ is hydrogen, methyl or ethyl, or
R⁵, R⁶ and/or R⁷ are phenyl which is optionally substituted up to 3 times, identically or differently, by halogen, cyano, (C₁-C₄)-alkyl or (C₁-C₄)-alkoxy, or
R⁵ is a radical of the formulae 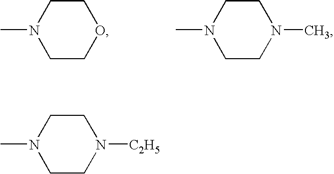
which may optionally be substituted up to twice, identically or differently, by chlorine or (C₁-C₄)-alkyl, or
R⁵ is a radical of the formula —NR⁹R¹⁰,
in which
R⁹ and R¹⁰ are identical or different and are hydrogen, (C₁-C₄)-alkyl or phenyl, or
R⁴ is carboxyl or is a radical of the formulae 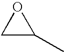
or —CO—R¹³ or O—R¹⁴
in which
R¹³ is (C₁-C₄)-alkyl,
R¹⁴ is (C₁-C₄)-alkyl which is optionally substituted up to 3 times, identically or differently, by hydroxyl or by a radical of the formula —NR¹⁵R¹⁶,
in which
R¹⁵ and R¹⁶ are identical or different and are hydrogen or (C₁-C₄)-alkyl which in turn may be substituted by phenyl, or
R⁴ is a radical of the formula —NH—CO—NR¹⁷R¹⁸,
in which
R¹⁷ and R¹⁸ are identical or different and are hydrogen or (C₁-C₄)-alkyl which is optionally substituted by hydroxyl, or
R¹⁷ and R¹⁸ form together with the nitrogen atom to which they are bonded a heterocyclic ring of the formulae 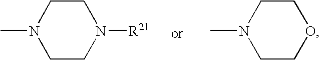
in which
R²¹ is hydrogen or (C₁-C₄)-alkyl, or
R¹⁷ and/or R¹⁸ are phenyl which is optionally substituted by chlorine, trifluoroethyl or by —SCF₃, or
R¹⁷ is hydrogen, and
R¹⁸ is a radical of the formula —SO₂—R²³, in which
R²³ is (C₁-C₄)-alkyl or phenyl which is optionally substituted by halogen, or is a radical of the formulae 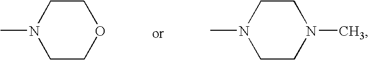
or
R⁴ is a radical of the formula
—NH—CO—R²⁴,
in which
R²⁴ is (C₁-C₄)-alkyl which is optionally substituted by phenyl which in turn may optionally be substituted by hydroxyl or (C₁-C₄)-alkoxy, or
(C₁-C₄)-alkyl is optionally substituted by a radical of the formula —(SO₂)_{b}—R²⁷, in which
b is either 0 or 1, and
R²⁷ is a radical of the formulae 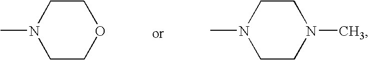
or
R⁴ is (C₁-C₆)-alkyl which is optionally substituted up to 3 times, identically or differently, by hydroxyl, phenyl or by radicals of the formulae —NR²⁸R²⁹ or —O—CO—R³⁰,
in which
R²⁸ and R²⁹ are identical or different, are hydrogen, phenyl or (C₁-C₄)-alkyl which is optionally substituted by hydroxyl, (C₁-C₄)-alkoxy or phenyl, or
R²⁸ and R²⁹ form together with the nitrogen atom to which they are bonded a heterocyclic ring of the formulae 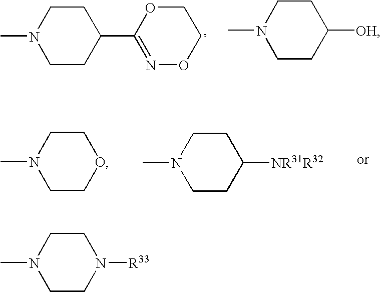
in which
R³¹ and R³² are identical or different and are hydrogen or (C₁-C₄)-alkyl,
R³³ is (C₁-C₄)-alkyl, benzyl, (C₁-C₄)-alkoxycarbonyl, (C₁-C₄)-alkylcarbonyl, carboxyl, pyridyl, pyrimidyl or phenyl which is optionally substituted by (C₁-C₄)-alkoxy, and
R³⁰ is (C₁-C₆)-alkyl, or
(C₁-C₆)-alkyl is optionally substituted by triazolyl which may in turn be substituted up to twice, identically or differently, by (C₁-C₄)-alkyl, where the latter may optionally be substituted by hydroxyl or (C₁-C₄)-alkoxy,
in which or
R⁴ is a radical of the formula —O—R³⁷, in which
R³⁷ is a radical of the formulae 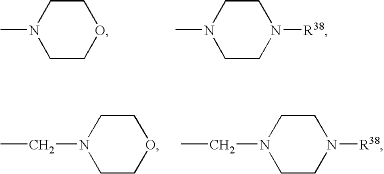
or —(CH₂)_{c}—NR³⁹R⁴⁰, in which
R³⁸ is hydrogen or (C₁-C₄)-alkyl,
c is either 0 or 1,
R³⁹ and R⁴⁰ are identical or different and are hydrogen or (C₁-C₄)-alkyl which is optionally substituted by hydroxyl, or
R⁴ is a radical of the formula 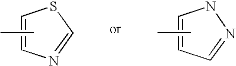
which is optionally substituted, in the case of pyrazole also via the N function, a total of up to 3 times, identically or differently, by trifluoromethyl or by phenyl which may in turn be substituted one or more times by chlorine or trifluoromethyl,
and/or is optionally substituted by cyclopentyl, cyclohexyl or by (C₁-C₆)-alkyl which may in turn be substituted by (C₁-C₄)-alkoxy, amino or by phenyl,
and/or may optionally be substituted by —NR⁴³R⁴⁴, —NH—CO—R⁴⁶, —NH—CO—CH₂—R⁴⁷ or —CO—R⁴⁸,
in which
R⁴³ and R⁴⁴ are identical or different and are hydrogen, benzyl, (C₁-C₄)-alkyl or phenyl which is optionally substituted by halogen or trifluoromethyl,
R⁴⁶ is (C₁-C₄)-alkyl or phenyl,
R⁴⁷ is hydroxyl or (C₁-C₄)-alkoxy,
R⁴⁸ is phenyl which is optionally substituted by chlorine, trifluoromethyl or (C₁-C₄)-alkoxy,
and the tautomers thereof, and the pharmaceutically acceptable salts, hydrates and prodrugs thereof.

4. The use method as claimed in claim 1 wherein the compounds has the following structures: 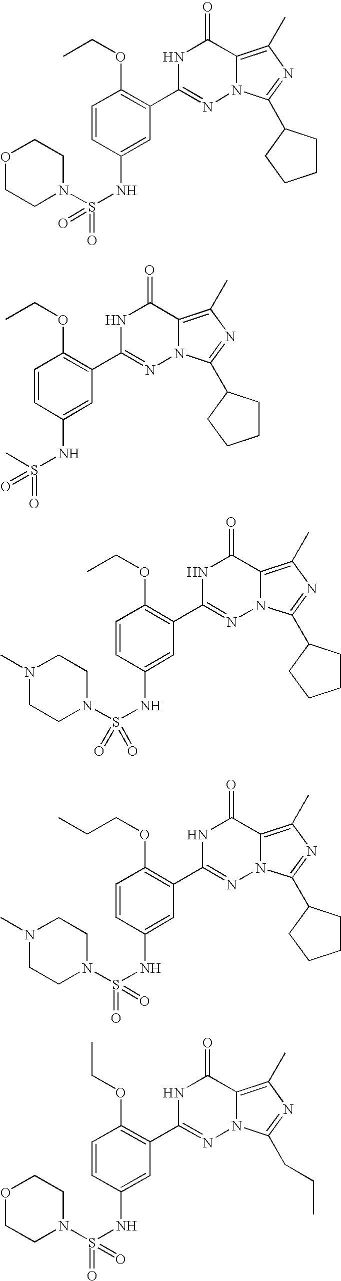 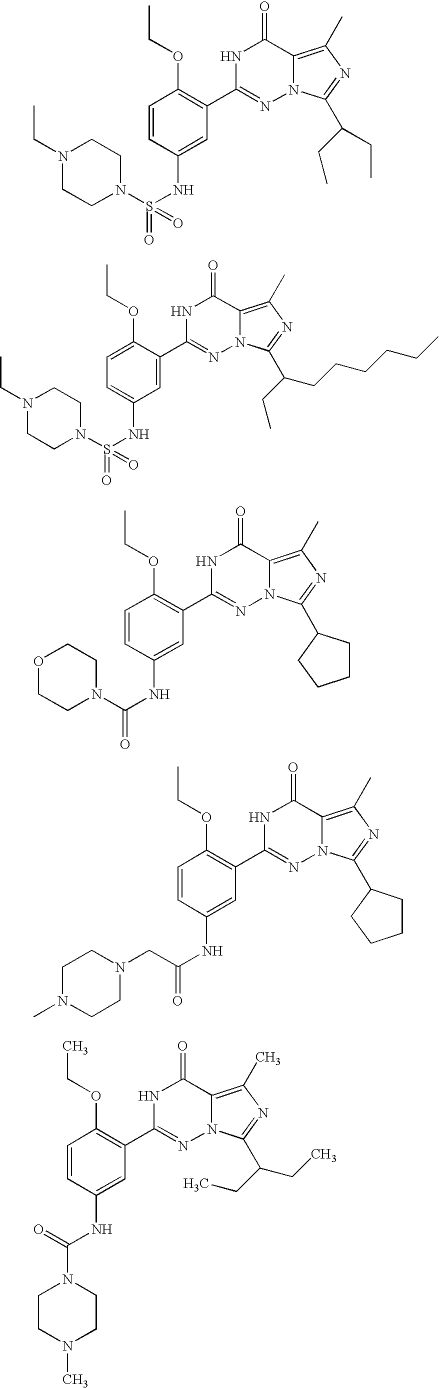 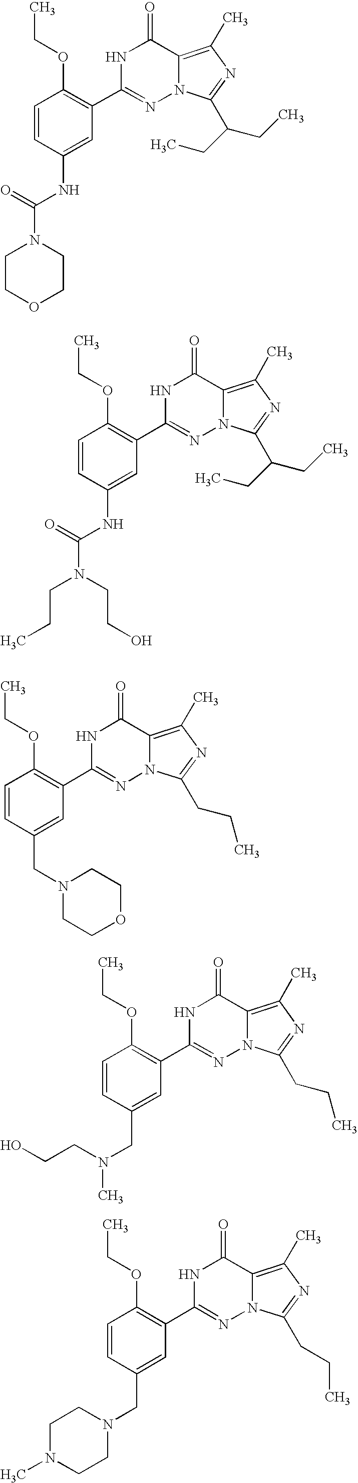 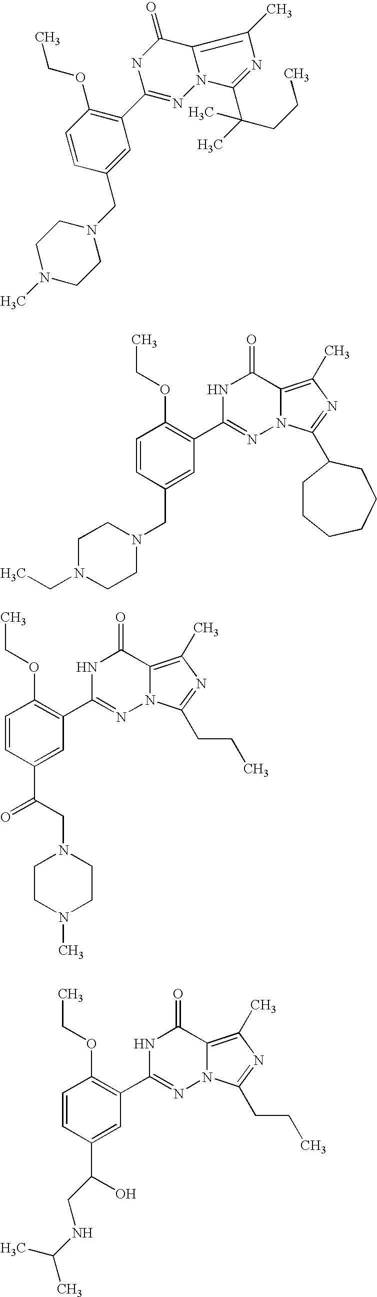 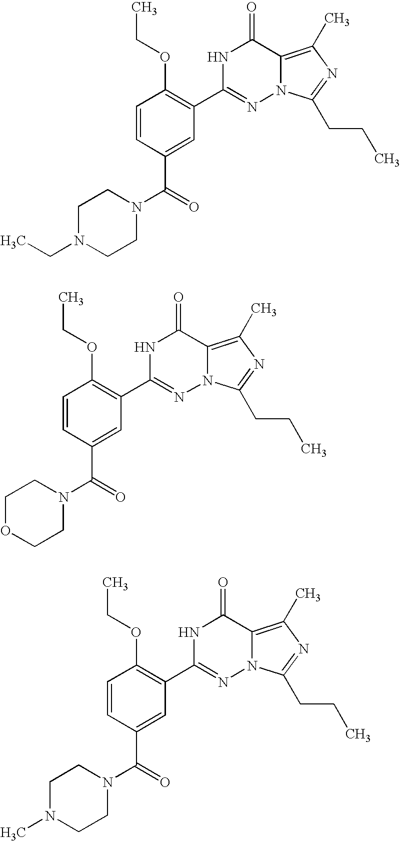
and the tautomers thereof, and the pharmaceutically acceptable salts, hydrates and prodtrugs thereof.

5. (canceled)

6. A method for the treatment of diseases in which an improvement and/or cure of the pathological condition can be achieved by improving the microcirculation of a tissue which comprises a cGMP-metabolizing phosphodiesterase comprising administering to a subject an effective amount of a compound of any one of claims 1 to 4.

7. A method for the treatment of and/or prophylaxis of coronary heart disease, heart failure, pulmonary hypertension, bladder disorders, prostate hyperplasia, nitrate-induced tolerance, ocular disorders, as glaucoma, central retinal or posterior cilliary arterial occlusion, central retinal venous occlusion, optic neuropathy, anterior ischemic optic neuropathy, glaucomatous optic neuropathy, of macular degeneration, diabetes, disorders of the peristalsis of stomach and esophagus, female infertility, premature labor, preeclampsia, alopecia, psoriasis, renal syndrome, cystic fibrosis, and cancer, comprising administering to a subject an effective amount of a compound of any one of claims 1 to 4.

8. A method for improving perception, concentration, learning and/or memory comprising administering to a subject an effective amount of a compound of any one of claims 1 to 4
